(19) United States
(12) Patent Application Publication
(10) Pub. No.: US 2006/0264799 A1
(43) Pub. Date: Nov. 23, 2006

(54) System and method for measuring the rate of flow of cerebral spinal fluid into an external ventricular drainage mechanism

(76) Inventor: Raymond J. Caluori, Quincy, MA (US)

Correspondence Address: MIRICK, O'CONNELL, DEMALLIE & LOUGEE, 100 FRONT STREET, WORCESTER, MA, 01608, US

(21) Appl. No.: 11/233,528

(22) Filed: Sep. 22, 2005

Related U.S. Application Data
(60) Provisional application No. 60/683,674, filed on May 23, 2005.

(51) Int. Cl. A61M 5/00

(52) U.S. Cl. 604/9

(57) ABSTRACT

A system and method of determining the rate of flow of Cerebral Spinal Fluid (CSF) in an External Ventricular Drainage Mechanism (EVDM). Drops, or the flow of CSF moving within or into the EVDM, are detected. The user is provided, in a human-readable form, information derived from the detection. This may be accomplished with a printer and/or a digital display device. The information may include CSF flow rate and/or CSF volume collected in the EVDM, for example. The system and method may further determine if the drops or flow have ceased for at least a certain amount of time. In that case, an alarm may be activated.

### CROSS REFERENCE TO RELATED APPLICATION

This application claims priority of Provisional application Ser. No. 60/683,674, filed on May 23, 2005.

### FIELD OF THE INVENTION

This invention relates to a system and method of measuring the rate of flow of cerebral spinal fluid into an external ventricular drainage mechanism.

### BACKGROUND OF THE INVENTION

A cerebral shunt works by regulating the pressure at which Cerebral Spinal Fluid (CSF) is allowed to leave the brain and taken to another part of the body for absorption. The brain produces approximately 18 ml/hour of CSF. Total CSF in circulation in an adult is approximately 140 ml, with 25 ml in the ventricles and 115 ml in the subarachnoid space. Cerebral shunts are placed in patients who have either an inability to absorb CSF, or when flow of CSF from the ventricles of the brain is obstructed, causing CSF that should flow from the brain to be trapped. Trapped fluid increases the Intra-Cranial Pressure (ICP). This increased pressure, if not released, can have many negative effects on the patient's brain that can cause injury or even death.

Normally in a shunted patient, a differential pressure valve that is placed externally on the skull controls the pressure at which CSF will be permitted to leave the brain. The CSF is released by the differential pressure valve at a certain pressure considered to be in the normal pressure range. This normal range of pressure in the cerebellum is usually from 5 mm Hg to 22 mm Hg. A catheter or tube that is in a ventricle of the brain passes through the skull and is connected to this differential pressure valve. On the other side of this valve another catheter is connected; this second catheter brings the CSF that has passed through the valve to another place in the body to be absorbed, usually the peritoneum, pleura or atrial cavities. These are the typical locations where the CFS is finally absorbed for a patient that is shunted. The pressure differential valve may incorporate a manual pump that can be used to manually pump CSF from the brain, and an anti-siphoning mechanism to keep excess CSF from leaving the brain. Having a “Shunt” or being “Shunted” is commonly how a patient is referred to who has had this apparatus placed in their brain.

External Ventricular Drainage Mechanisms (EVDMs) are bedside devices that are used in two typical circumstances. The first is for patients who have had a Ventriculo-Peritoneum, Ventriculo-Pleura, or Ventriculo-Atrial shunt externalized. The reason for the shunt externalization may be, e.g., injury, infection or some obstruction of the flow of CSF, requiring the shunt system to be removed temporarily from the patient's brain to allow healing of the injury, infection or for an obstruction to be removed. The time of externalization may vary before a shunt system can be installed again. During this time of externalization, a patient has an EVDM connected in place of the shunt system to control ICP. The purpose of the EVDM is to both control the ICP and measure the volume of CSF produced by a patient, usually measured hourly and daily.

EVDMs are typically adjusted in height relative to the patient's head position in order to maintain proper ICP, while at the same time allowing CSF to flow from the brain without allowing the removal of too much CSF. The position of the EVDM relative to the patients head is set avoid column pressure and column vacuum in the drainage tube: if the EVDM were positioned above the head, column pressures would increase, causing the need for more pressure for the CSF to leave the brain, which would create higher ICP, and possible patient discomfort or injury. If the EVDM were positioned below the head, this could cause a lower vacuum pressure in the drainage tube, resulting in the removal of too much CSF from the brain, which can cause low ICP and possible patient discomfort or injury. The positioning of the EVDM next to the mid point of the head around the ear area usually is done while the patient is not moving, usually in their bed. The apparatus elevation is adjusted on a sliding bar that is typically in increments of mm Hg and/or cm H₂O to control the ICP. This scale is used to adjust the ICP that is required for the patient. A collection tube is attached on a sliding bar that allows the tube to be raised or lowered to a level that will create the correct ICP.

There are problems with the monitoring of CSF in the current usage of EVDMs. One problem is there is no alarm. The typical EVDM in use today has a manual on/off valve that provides on/off CSF flow control into the collection apparatus. When a patient needs to be moved for short periods of time, for example when being transported for any testing or diagnostic work like X-rays, MRIs or simply to go to the bathroom or stand up, to avoid siphoning off too much CSF from a patient's brain the on/off valve is typically turned off by the caregiver. Once the patient's activities have stopped and the patient is settled back into a lying or stable position, the caregiver reopens the valve or valves to allow the CFS to resume its flow from the brain. In the business of resettling the patient to a physically stable position to resume the CSF drainage, the caregiver can easily forget to reopen the valve or valves to allow the CSF to resume its flow. If the valve(s) are not reopened, the CSF is held in the brain, causing the ICP to begin to rise above normal. This can lead to discomfort, brain injury and ultimately death by crushing the brain if not caught in time.

Another problem with EVDMs is how the volume and rate of flow of CSF from a patient's brain are determined. Currently, EVDMs have a CSF collection apparatus, typically a clear tube marked in increments of cubic centimeters and fractions of centimeters, that the caregiver looks at to determine how much CSF has been collected. The caregiver then has to manually calculate the volume and rate of flow of CSF from that patient during a given time period. The time periods that are usually recorded are hourly. This can be very hard to monitor accurately because typically the caregiver is a nurse who is caring for several patients at the same time, making it difficult for the nurse to be present to take measurements right on the hour. This leads to inaccuracies in the monitoring and recording of the actual CSF flow rate and collection volume.

### SUMMARY OF THE INVENTION

The invention features a volume measurement and/or alarm system for the collection of Cerebral Spinal Fluid (CSF) into External Ventricular Drainage Mechanisms (EVDMs). The system accomplishes one or both of two major functions. The first function is to measure the rate of flow of CSF draining from a patient through an external cerebral ventricle shunt over a given time period (e.g. per hour). The second function is to notify the caregiver (e.g. with an audio and/or visual alarm) if the CSF stops flowing for a given interval of time; this given interval of time can be programmable/adjustable by the operator.

The inventive monitoring system will also apply to cases where partial externalization of the shunt has been accomplished. This is where the shunt system is left in the patient's brain and only the distal end of the shunt drainage tube is removed from the drainage/absorption point in the patient's body and placed into the EVDM. The invention can also be used for patients who have a cerebral shunt system implanted temporarily for evaluation, after injury, surgery or infection, for example.

The inventive measurement and alarm system is designed to monitor and display electronically the rate of flow of CSF, and volume produced during a given time period, usually measured hourly, and to audibly and/or visually alert caregivers if the flow of CSF into the collection apparatus of the EVDM stops or is interrupted for a given time period that may vary from a few seconds or minutes, to as much as several hours. This interval of time before the alarm is turned on may be adjustable/programmable by the operator.

The monitor preferably reads the rate of flow of CSF through a visually clear or non-clear structure or tube wall, so no direct contact is made with the sterile CSF, which serves to avoid any possibility of contamination and/or infection. The monitor may alternatively measure the volume of CSF collected in the EVDM's CSF collection apparatus. The monitor is preferably accomplished with an optical, mechanical, and/or ultrasound-based measurement system. Preferably, the system measures fluid flow to, through, or in the EVDM. Other feasible methods for fluid flow detection and/or volume of fluid measuring could be used, as described below.

The alarm may be electrically powered or battery powered, or electrically powered with a battery back up. The battery back up will allow caregivers and patients to disconnect the measurement/alarm system from a wall socket and still operate on the battery for some period of time. This alarm device will allow the medical caregivers to know when the manual on/off valve may have been left in the off position by sounding after a predetermined time period.

The alarm can alert caregivers and the patient in any variety of ways. It may be an audible and/or visual-type alarm. The alarm can alert in a steady or interval fashion, both audibly and/or visually. The auditory and visual may be synchronized where they come on together or where one comes on first and at a later interval the second one comes on. This alarm may be built into the EVDM, or installed in addition to an EVDM. The alarm may be connected to an additional remote or wireless alarm system. The alarm may have a reset button that can be reset immediately by the operator when the alarm sounds, starting the alarm cycle again.

The CSF flow needs to be maintained in a sterile environment, which requires some form of reading the fluid through the structure through which the CSF moves and/or is collected or contained in after collection, or in the process of traveling to or through the EVDM.

In a preferred embodiment, the inventive monitoring system includes a photo emitter and photo detector that together determine the number of drops of CSF that flow or drip into the EVDM's collection apparatus. As the chemical make-up of CSF is known, in any given EVDM, the drops will have a known, consistent volume. Optionally, a calibration capability may be included in the system to allow the operator to visibly count or otherwise determine the number of drops per cubic centimeter or the like, and manually program that number into the computer to allow accurate readings for each individual patient. This would make up for any difference in CSF surface tension, specific gravity or chemical nature, or differences in the EVDM collection apparatus, any of which could affect the volume of a drop of CSF. This allows the measurement/alarm's computer to calculate and digitally display in real time the rate of flow of CSF the patient is producing, based on the number of drops counted by the system. The computer can also display the volume collected in or over a certain increment of time. The volume of CSF produced is usually recorded in volume per hour. This monitor system may also incorporate a function to cumulatively display the total volume of CSF for the entire externalization period, which may be days, weeks or months. It may also display the past history of the collection for a given time period going back in increments of time. For example, if a caregiver needs to know the rate and volume of CSF collected hourly for the past number of hours, days, or weeks, that historical information may be stored onboard and displayed. A printer may also be incorporated to this design. The printed record may have all the information that would be digitally displayed on the monitor. A paper read out of the CSF collected may also be printed with the rate and volume of CSF collected for a given increment of time. This increment of time may, for example, be hourly, on the hour for accurate record keeping, or some other programmable increment of time. The system may be programmed to automatically print on the hour or some other increment of time, and/or manually print CSF collection records on demand. The on demand printing functionality may be designed to work even while the automatic printing option is employed. Both time and date information would be displayed digitally on the monitor display and on all printed records.

This invention features a system and method of determining the rate of flow of Cerebral Spinal Fluid (CSF) in an External Ventricular Drainage Mechanism (EVDM), comprising detecting drops or the flow of CSF moving within or into the EVDM, and providing to a user in a human-readable form, information derived from the detection. The information may comprise CSF flow rate and/or CSF volume collected in the EVDM, for example. The system and method may further comprise determining if the drops or flow have ceased for at least a certain amount of time. In that case, an alarm may be activated.

### BRIEF DESCRIPTION OF THE DRAWINGS

Other objects, features and advantages will occur to those skilled in the art from the following description of the preferred embodiments, and the accompanying drawings, in which like numbers are used for like parts, and in which:

FIGS. 1A and 1 B are front and top schematic views, respectively, of the preferred embodiment of the invention that employs a light-based system for counting the drops of CSF dripping into the EVDM collection tube;

FIG. 1C is a schematic diagram of the preferred embodiment of the system of this invention; and

FIGS. 2 through 10 are schematic diagrams that illustrate the several alternative manners of detecting CSF drops and/or flow for use in the invention.

### DETAILED DESCRIPTION OF THE PREFERRED EMBODIMENTS

The disclosure of Provisional application Ser. No. 60/683,674, filed on May 23, 2005 is incorporated herein by reference.

There are two main purposes of the inventive CSF measurement/alarm. First, to measure accurately and display digitally and in printed form, the rate of flow of CSF, and the volume of CSF produced by a patient. Secondly, to determine when CSF is no longer flowing at a predetermined rate and for a predetermined period of time, or has been interrupted for a time period that is greater than the time that has been programmed for the alarm. The interval of time when the alarm will go off is preferably determined by and input into the system by the operator/caregiver, and can be from a few seconds, to minutes or hours. An additional purpose of this device is to have a digital display that reads out the rate of CSF being produced and the volume of CSF collected hour-by-hour, or some other increment of time programmed by the operator. A failsafe may be built in such that the alarm is activated if any active component fails.

This invention may be accomplished in a CSF flow monitoring, measurement and alarm system in which a photo emitter and a photo detector are attached externally to the CSF collection tube currently used by most External Ventricular Drainage Mechanisms (EVDMs). Alternatively, the photo emitter and a photo detector can be attached to a drip chamber, other than the collection tube currently used in EVDM's, created by interrupting the conduit carrying the CSF from the patient to the EVDM. The emitter shines a light through the tube in such a fashion that the light shining through the tube is in the direct path of the dripping CSF being collected. The light is emitted from one side of the collection tube and is read by a photo detector on the other side of the collection tube. The detector is sensitive to the drops of CSF falling through the light path created by the emitter. By determining the frequency at which the drops fall into the collection tube, an accurate determination of the number of drops, and of when drops of CSF stop dripping into the collection tube or container can be made. The alarm time can be programmed by the medical operator, or be pre-set in the device. When the dripping stops for this time period, an alarm will activate, usually light and/or sound. This alarm may have a reset button that can be reset immediately by the operator when the alarm sounds, to start the alarm cycle again.

This monitor system may incorporate a function to display and/or print the flow rate, the total volume of CSF collected for a given period (e.g. per hour), and the cumulative volume for the entire externalization period, which may be days, weeks or months. It may also display/print the past history of the collection for a given time period going back in increments of time. For example, if a caregiver needs to know the rate and volume of CSF collected hourly for the past number of hours, days, or weeks, that historical information may be stored onboard and displayed/printed. A printer may also be incorporated to this design. The printed record may have all the information that would be digitally displayed on the monitor. A paper read out of the CSF collected may also be printed with the rate and volume of CSF collected for a given increment of time. This increment of time may, for example, be hourly, on the hour for accurate record keeping, or some other programmable increment of time. The system may be programmed to automatically print on the hour or some other increment of time, and/or manually print CSF collection records on demand. The on demand printing functionality may be designed to work even while the automatic printing option is employed. Both time and date information would be displayed digitally on the monitor display and on all printed records.

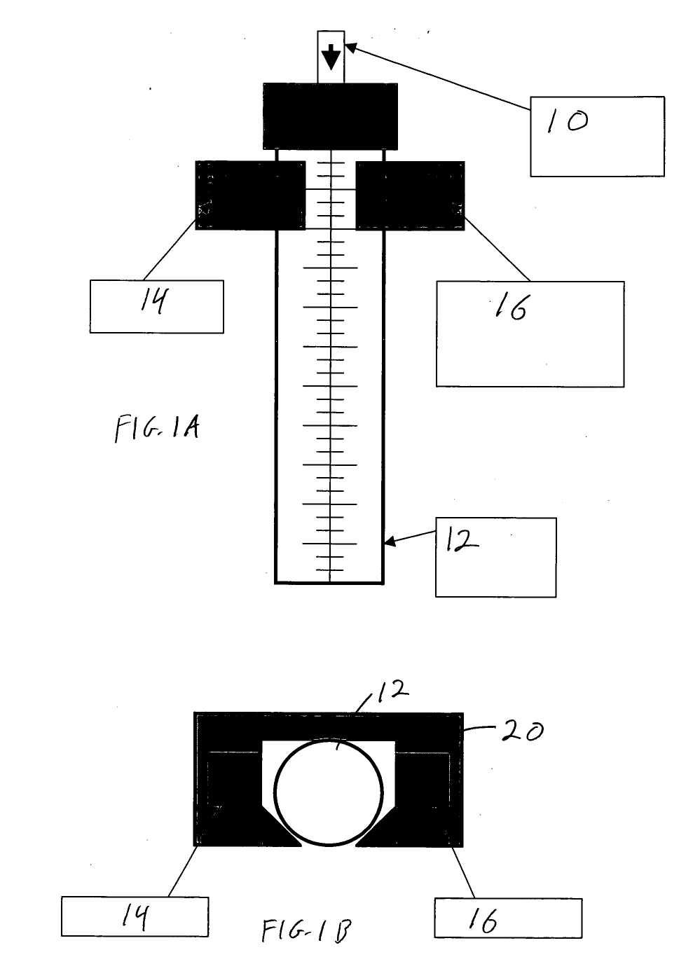

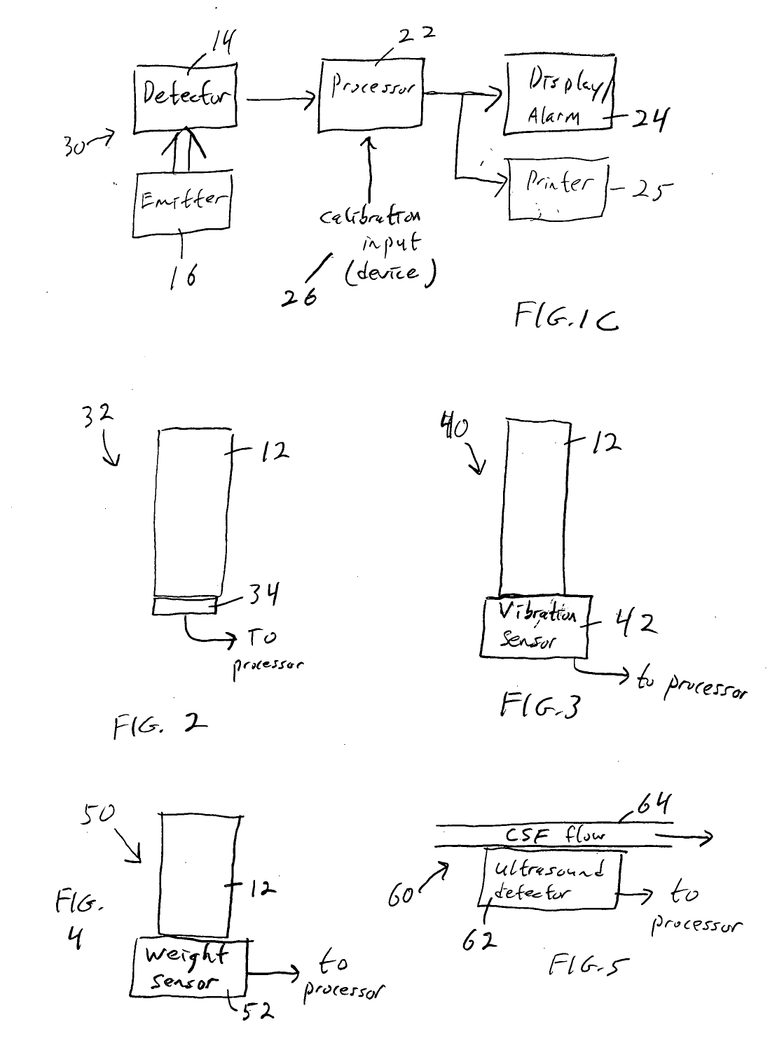

In the preferred embodiment of the invention (FIGS. 1A, 1B and 1C), system 30 includes a photo emitter 16 (e.g. an LED) and a photo detector 14 are placed on opposite sides of an optically-clear portion of a structure through which the CSF moves (e.g. graduated collection tube 12), and the drops of CSF are counted as they pass through the light beam. Structure 20 holds tube 12 in place. Tubes 10 and 12 and structure 20 are part of an EVDM.

The photo emitter will shine a light emission directed across the path of the dripping CSF. CSF enters tube 12 through tube 10. The photo receptor or detector, placed opposite the photo emitter, and facing the photo emitter so that it detects the light emitted by the emitter, is then able to detect variations in the amount of light caused as a drop of CSF moves through the light beam; as a drop of CSF falls through the light field created by the photo emitter, the photo detector output changes. This change is indicative of the interference created by a drop of CSF by falling through the light field.

A microprocessor 22 senses these variations of light, and from the drop count can calculate the CSF flow rate, and total volume of CSF leaving a patient's brain over a given period of time. Appropriate outputs are displayed on display/alarm 24, and/or printed on printer 25. If the processor does not sense any variances of light for a given period of time, an alarm will activate, typically light and/or sound to alert the caregiver.

Some of the advantages that make this the preferred design are:

a.) Light will shine through the clear material of a vial, collection apparatus, or interruption chamber, allowing an accurate read of CSF with no physical interference to its flow path into the EVDM.

b.) This non-mechanical method also eliminates the possibility of a mechanical failure that could lead to inaccurate readings.

c.) This non-mechanical method also eliminates the possibility of clogging, stopping or slowing the flow of the CSF through the conduit leading from the patients brain resulting in a rise in ICP.

d.) The CSF remains in a sterile environment.

e.) Ambient light will not interfere with the light field the CSF drop falls through.

f.) Light will not alter or affect the chemical make up of the CSF.

g.) Light will not alter or affect any foreign bodies in the CSF, such as viruses or bacteria, which are commonly and routinely tested for when patients have externalized shunts. If any other kind of a field, such as a high frequency field or electric current were used, the field could kill a virus or bacteria, which could give a false test result that could be fatal if the patient's brain is actually infected and goes untreated due to a false negative reading.

h.) Light does not create an electronic, high frequency, or magnetic field that could alter the chemical make up of the CSF or any foreign bodies in the CSF.

i.) Light does not create a field that could be affected by human handling, objects or metal passing close to the light sensors. Handling of, or objects passing close to other type fields such as, high frequency, electrical current, magnetic, or other fields, could affect or interfere with those fields resulting in inaccurate reading by the sensors and may yield inaccurate readings of the rate and flow of CSF.

j.) Light is not affected by the typical handling, movement, vibration or bumping of the EVDM or light sensors in a hospital room, or when the patient is being moved or transported.

k.) This is the most cost effective and proven technology that can be used.

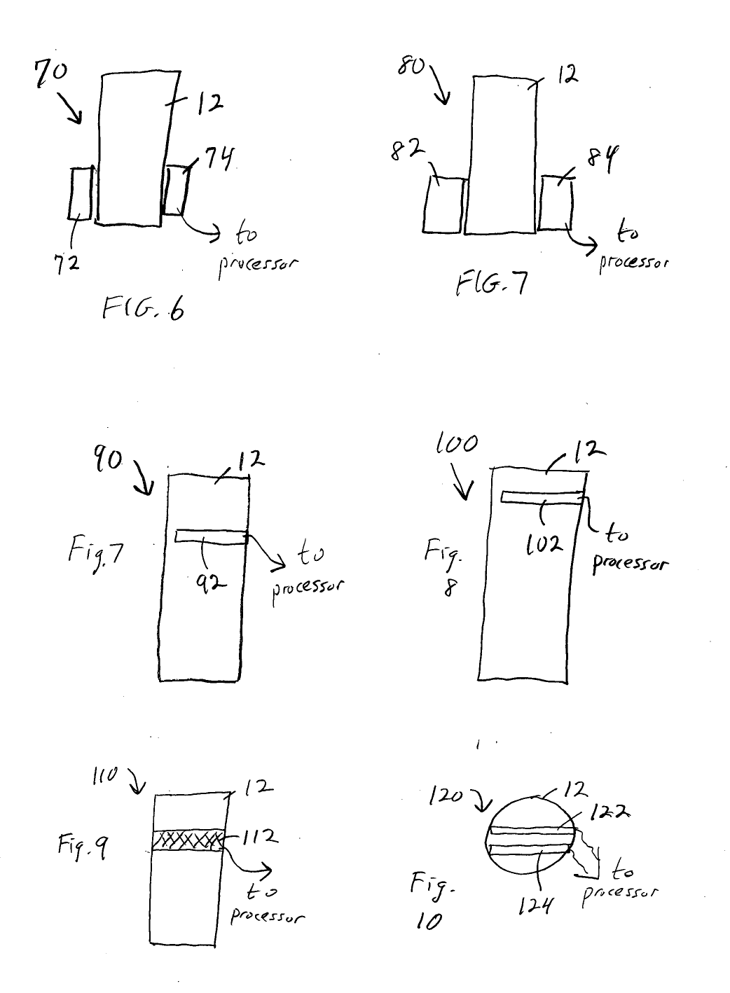

Several alternative embodiments of the invention are contemplated within the scope of the invention, and described below. These include a capacitance-based sensor 32, FIG. 2, that has one or more conductive plates 34 attached to the CSF collection apparatus 12. The plate(s) are excited so that the capacitance of the fluid volume can be determined. Each additional drop of CSF that falls into the collection apparatus increases the volume of CSF, which in turn changes the capacitance of the volume of CSF. Every time the volume of a drop of CSF is added to the collected volume of CSF, a slight proportional change of capacitance will occur. The microprocessor senses the change in capacitance and calculates the flow rate and volume of CSF. If there is no change to capacitance, and therefore the volume of CSF, for a given period of time, the alarm will activate.

This arrangement would allow the CSF to always be in a sterile environment and have no interaction with anything outside of the sterile interior of the EVDM.

Yet another alternative embodiment 40, FIG. 3, is based on vibration. A drop of CSF falling into a collected volume in the EVDM collection apparatus (e.g. collection tube 12) causes a vibration in the volume. A sensor 42 that is excited with every drop that falls into the fluid (e.g. a pressure sensor) is connected to a microprocessor that counts the vibrations and calculates the rate and flow of CSF during a given period of time. If no vibrations are detected for a given period of time an alarm will activate.

This arrangement would employ a sensor that is on the inside of the tube, but with leads passing through the tube, the CSF will remain sterile. This method would allow the CSF to always be in a sterile environment and have no interaction with anything outside of the sterile interior of the EVDM.

Yet another alternative embodiment 50, FIG. 4, works by detecting a change in mass of the collected CSF volume. Every time a drop of CSF falls into the collection tube 12, the total weight of the tube plus collected CSF increases. In other words, this arrangement would sense that the whole collection tube just gotten heavier. The weight is determined by sensor 52, which could be a scale or strain gauge, for example. A microprocessor then calculates the flow rate and total volume of CSF collected from the weight of the CSF that has been collected, and the weight change over time. If no weight change is detected for a given period of time, an alarm will be activated.

This arrangement would employ sensors that are on the exterior of the sterile EVDM collection container. This method would allow the CSF to always be in a sterile environment and have no interaction with anything outside of the sterile interior of the EVDM.

An alternative weight-based arrangement contemplates detecting small changes in weight on a drop-by drop basis, not total weight. In this case, the weight sensor detects a momentary change in weight when a drop of CSF falls into the collection apparatus or tube; the weight sensor will detect the momentary “tap” or increase in weight, and a microprocessor will calculate the CSF flow rate by counting the number of momentary weight changes detected in a given period of time. The total volume can then be determined from this information. If no weight changes are detected during a given time period, an alarm will activate. This method would employ sensors that are on the exterior of the sterile EVDM's collection container. This method would allow the CSF to always be in a sterile environment and have no interaction with anything outside of the sterile interior of the EVDM.

Still another alternative arrangement 60, FIG. 5, uses ultrasound to determine flow in a tube 64. The movement of CSF flowing through tube 64 is determined by reflective sound waves using ultrasound detector device 62. This can be sensed at various locations, such as the EVDM tubing or some other conduit. A microprocessor can be used to calculate the speed of the CSF flowing through a conduit, and from this can calculate the CSF flow rate and total collected volume. Volume can also be determined by a sonic change detected in the volume of fluid that is collected, for example in collection tube 12, FIG. 1A. If the system does not detect a movement in the collection conduit for a given period of time, an alarm will activate.

In a related embodiment, sound waves are used to determine the volume of CSF in a collection vessel such as collection tube 12, and flow rate can be determined based on the rate of change of this volume. If there is no change in the volume of CSF for a given period of time an alarm will sound.

Another embodiment 70, FIG. 6, uses changes in resistance. There are two ways to employ this arrangement. In a first method, every time the volume of a drop of CSF is added to the collected volume of CSF in tube 12, a slight change in the electrical conductivity, or resistance, of the total volume of fluid will be detected. Electrodes 72 and 74 are used for this purpose. With each additional volume (drop) of CSF added to the total volume collected, the conductivity will change. Again, a microprocessor can from these readings calculate the CSF flow rate and total collected volume. If there are no resistance changes sensed to the collected volume of CSF for a given period of time, an alarm will activate. This method may employ sensors and leads that are on the interior or exterior of the sterile EVDM's collection container. This method would allow the CSF to always be in a sterile environment and have no interaction with anything outside of the sterile interior of the EVDM.

In a related alternative, this same methodology is used to determine the volume of fluid collected. Every time a new drop of CSF falls into the collected volume, a momentary slight change in the electrical conductivity, or resistance, of the total volume of CSF is measured. A microprocessor will calculate the total number of resistance changes and equate this to the number of drops added, and calculate and display the flow rate and total volume of CSF collected during a given period of time. If there are no resistance changes sensed for a given period of time, an alarm will sound. This method may employ sensors and leads that are on the interior or exterior of the sterile EVDM's collection container. This method would allow the CSF to always be in a sterile environment and have no interaction with anything outside of the sterile interior of the EVDM.

Still another alternative embodiment 80, FIG. 7, employs a magnetic field that is created around either the containing apparatus 12 that the fluid collects in, or the conduit leading to the collection apparatus. The magnetic field is created by magnet 82 and sensed by sensor 84. The field is positioned in such a way that the drops of CSF have to pass through the magnetic field. Each time a drop of CSF passes through the magnetic field, the magnetic field changes slightly. A processor will calculate the number of changes/disturbances to the magnetic field to determine the rate and volume of CSF collected for a given period of time. This method may employ sensors and leads that are on the exterior of the sterile EVDM's collection container. This method would allow the CSF to always be in a sterile environment and have no interaction with anything outside of the sterile interior of the EVDM.

In a related alternative, this same methodology is used to determine the volume of fluid collected. A magnetic field will be created around the containing apparatus 12 that the CSF collects in. The magnetic field will be positioned in such a way that as drops of CSF collect in the collection apparatus there will be a change to the magnetic field of the entire collected volume of CSF. The processor calculates the number of magnetic changes/disturbances to the collected volume of CSF to determine the flow rate and volume of CSF collected for a given period of time. This method may employ sensors and leads that may be on or molded into the exterior of the sterile EVDM's collection container. This method would allow the CSF to always be in a sterile environment and have no interaction with anything outside of the sterile interior of the EVDM. If no change in the magnetic field is detected for a given period of time an alarm will activate.

Yet another arrangement 90, FIG. 8, uses a mechanical interruption to the flow of CSF to count drops. As the CSF drips into the collection apparatus 12, the drops fall onto a small flexible or jointed mechanical structure 92 placed into the flow path. The drop of CSF will move the mechanical structure. The movement of this mechanical structure can be detected in any one of possible known ways, such as those described above, or by arranging a switch that is opened or closed as the structure moves. A processor will calculate the number of movements to determine and display the flow rate and volume of CSF collected for a given period of time. If no movements are detected for a given period of time an alarm will sound. This method may employ sensors and leads that are on the exterior of the sterile EVDM's collection container. This method would allow the CSF to always be in a sterile environment and have no interaction with anything outside of the sterile interior of the EVDM.

In another alternative arrangement 100, FIG. 8, the mechanical structure 102 that is impacted by the CSF drops has an electrical detection structure/device (such as resistance or capacitance-based sensing) built into structure 102 and any necessary supporting devices, such as powers sources (not shown). The CSF drips on the physically interfering protrusion 102 before continuing its path into the collection container 12. This structure that momentarily interferes with the falling path of a drop of CSF can assume any shape. As an example, it may be shaped like a finger or any other shaped protrusion that will be attached to the wall of the collection apparatus and will momentarily obstruct the CSF path so that the CSF will contact the protrusion and then continue on a path into the collection apparatus. This physically interfering structure to the falling drip of CSF will have some fashion of conductive material, metal or some other type of material, depending on the sensing method to be employed, that will be charged electrically or with high frequency. When the drop of CSF makes contact with the protrusion it will momentarily sense a corresponding resistance or capacitance change/fluctuation. A processor will detect the number of changes and calculate the flow rate and volume of CSF collected for a given time period. If no change is sensed by the processor for a given period of time an alarm will activate.

In addition, any or all of the above sensing methods may be employed to “charge” or activate the protruding interfering structure 102, allowing a sensor to be stimulated that is connected to a processor that will read any changes. Whether magnetic field, capacitance, resistance, Doppler, weight change, vibration, pressure, or any other form of sensing contained in this patent application. This protruding interfering structure to the path of the dripping CSF may be rigid or flexible. Because it can be molded into the collection container, the sensing leads can be connected to the sensor without having the sensor inside the collection container. This would allow the CSF to always be in a sterile environment and have no interaction with anything outside of the sterile inside of the EVDM.

An alternative to this arrangement, system 110, FIG. 9, contemplates a screen type conductive structure 112, or some other type material depending on the sensor method used to detect a CSF drip, or another mesh-type material in which the space created by the mesh is smaller than a drop of CSF, so that a drop of CSF falling into tube 12 cannot pass though the mesh material without making contact with it. A sensor detects a drop of CSF has passed through the screen, and the processor calculates the flow rate and volume of CSF by the number of drops that have fallen through the screen type material. Any of the sensing methods listed above can be employed with this arrangement, such as pressure, vibration, capacitance, resistance, weight, and sound. If no change is sensed by the processor for a given period of time, an alarm will activate.

Another similar alternative 120, FIG. 10, contemplates using two leads 122, 124 that are positioned very close to each other. The leads may be rigid or flexible. The space between the leads will be some distance less that one quarter of an inch. Preferably the space between these leads is much smaller than the diameter of a drop of CSF. The leads are oriented so that when a drop of CSF falls into tube 12, the drop it will contact both leads. The two leads' make up will be determined by what method of sensing is used, whether electrical conductivity, magnetic field change, resistance, capacitance, pressure, vibration, weight or motion. Depending on the method of sensing the two leads' construction and arrangement may be different. These two leads may consist of two pins, or two pieces of metal or material that are spaced by a small gap less that one quarter of an inch apart. The leads would typically be less than 24 inches in length. The leads may be a series two or more wires made of metal or some other material, which are spaced closer together than the diameter of a drop of CSF and oriented in such a fashion that each drop of CSF will contact two (or more if there are more than 2 lead) of the wires as it drips past them. The processor will calculate the number of changes detected by the sensors, using whatever sensing method is employed with this design, to give the flow rate and volume of CSF for a given period of time. If no change is sensed by the processor for a given period of time, an alarm will activate.

Although specific features of the invention are shown in some drawings and not others, this is for convenience only as features may be combined as would be apparent to those skilled in the art, in accordance with the invention.

Other embodiments will occur to those skilled in the art and are within the following claims.

What is claimed is:

1. A method of determining the rate of flow of Cerebral Spinal Fluid (CSF) in an External Ventricular Drainage Mechanism (EVDM), comprising:
detecting drops or the flow of CSF moving within or into the EVDM; and
providing to a user in a human-readable form, information derived from the detection.

2. The method of claim 1, wherein the information comprises CSF flow rate.

3. The method of claim 1, wherein the information comprises CSF volume collected in the EVDM.

4. The method of claim 1, further comprising determining if the drops or flow have ceased for at least a certain amount of time.

5. The method of claim 4, further comprising activating an alarm if the drops or flow have ceased for at least a certain amount of time.

6. A system for determining the rate of flow of Cerebral Spinal Fluid (CSF) in an External Ventricular Drainage Mechanism (EVDM), comprising:
means for detecting drops or the flow of CSF moving within or into the EVDM; and
means, responsive to the means for detecting, for providing to a user in a human-readable form, information derived from the detection.

7. The method of claim 6, wherein the information comprises CSF flow rate.

8. The method of claim 6, wherein the information comprises CSF volume collected in the EVDM.

9. The method of claim 6, further comprising means for determining if the drops or flow have ceased for at least a certain amount of time.

10. The method of claim 9, further comprising means for activating an alarm if the drops or flow have ceased for at least a certain amount of time.
